(19) United States
(12) Patent Application Publication
(10) Pub. No.: US 2017/0295788 A1
(43) Pub. Date: Oct. 19, 2017

(54) USE OF HETEROCYCLIC FLUOROALKENYL SULFONE COMPOUNDS FOR REPELLING MOLLUSCS

(71) Applicant: Adama Makhteshim Ltd., Beer-Sheva (IL)

(72) Inventor: Dan KARMON, Gan HaShomron (IL)

(21) Appl. No.: 15/511,660

(22) PCT Filed: Sep. 17, 2015
(86) PCT No.: PCT/IL2015/050943

§ 371 Date: Mar. 16, 2017

Related U.S. Application Data
(60) Provisional application No. 62/051,968, filed on Sep. 18, 2014.

(51) Int. Cl. A01N 43/78; C07D 277/36

(52) U.S. Cl. CPC A01N 43/78; C07D 277/36

(57) ABSTRACT

Compounds of formula (I) in which X represents halogen, and n represents 0, 1 or 2, has been found to act as a repellent of molluscs such as slugs and snails.

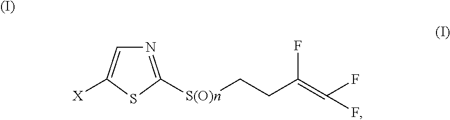

### TECHNICAL FIELD

The present subject matter relates to the novel use of compounds of Formula (I) for protecting plants from damage caused by snails and slugs.

### BACKGROUND OF THE PRESENT SUBJECT MATTER

Terrestrial gastropod molluscs such as slugs, snails, etc. are major pests of horticultural and agricultural crops in many parts of the world and whose numbers increase by feeding off of these crops. Molluscs cause severe damage by feeding, and also generate unsightly mucus trails. They are considered to be serious pests of leaf vegetables, fruits, root crops, flowering plants, cereals such as wheat, barley, oats and oil seed rape, rice and also of ornamentals etc. Molluscs feed both above and below the surface of the ground, on seeds, seedlings and plants. The can damage shoots, roots, leaves, flowers and fruits therefore reducing plant stand and crop yield.

Slugs are a major agricultural pest causing significant crop damage because they bury themselves in the soil and eat the inside of the seeds thereby destroying the crop. As discussed in WO 2006/108553, changes in the management of crops may lead to increased population densities of molluscs thereby resulting in further damage to the crops. These changes may include minimum tillage, direct drilling, and high organic matter build up in the soil. Slugs feed vigorously above and below the surface on seeds, growing points, shoots and roots along with other organic matter they can find.

In order to control slugs, snails, etc., a variety of extermination methods have previously been proposed. Current forms of molluscicides are generally in the form of pellets. These usually deteriorate upon exposure to water and in particular, rain and are not sufficiently durable as long-term molluscicides (Molluscs as Crop Pests, G. M. Barker, Mar. 21, 2002, Technology and Engineering).

The most commonly used active ingredient in molluscicides is metaldehyde, which disrupts the gastric organs of slugs and snails, causing death. Alternatives to chemical molluscicides include using traps or barriers, or designing gardens that are less attractive to slugs. Slug and snail baits containing iron phosphate as the active ingredient are also available. These molluscicides provide adequate levels of control/repellency.

As such, there is an unmet need for a highly efficient and safer mollusc repellent.

### SUMMARY

According to one aspect, the present subject matter provides a method of controlling or repelling molluscs. The method includes applying to a plant or soil in which the plants are planted an effective amount of a compound of Formula (I)

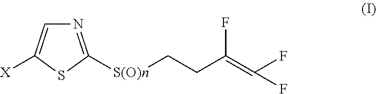

wherein
X represents halogen, and n represents 0, 1 or 2.

In some embodiments, X represents fluoro, chloro or bromo, and n represents 0 or 2. In a further embodiment, X represents chloro or bromo, and n represents 2. In an even further embodiment, X represents chloro, and n represents 2.

According to another aspect, the present subject matter relates to a mollusc repelling composition. The composition comprises a repellent amount of a compound of Formula (I)

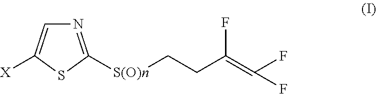

wherein X and n are as defined above.

According to another aspect, the present subject matter relates to the use of a compound of Formula (I)

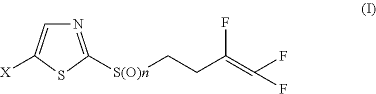

for controlling a mollusc population on plants or soil in which the plants are planted; wherein X and n are as defined above.

According to a further aspect, the present subject matter relates to a composition comprising an effective amount of a compound of Formula (I)

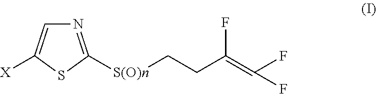

for use in controlling a mollusc population;
wherein X and n are as defined above.

### DETAILED DESCRIPTION

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

As used herein, the phrase “agriculturally acceptable carrier” means carriers which are known and accepted in the art for the formation of formulations for agricultural or horticultural use.

As used herein the term “plant” or “crop” includes reference to whole plants, plant organs (e.g. leaves, stems, twigs, roots, trunks, limbs, shoots, fruits etc.), plant cells, or plant seeds. This term also encompasses plant crops such as fruits. In yet another embodiment, the term “plant” may include the propagation material thereof, which may include all the generative parts of the plant such as seeds and vegetative plant material such as cuttings and tubers, which can be used for the multiplication of the plant. This may also include seeds, tubers, spores, corms, bulbs, rhizomes, sprouts basal shoots, stolons, and buds and other parts of plants, including seedlings and young plants, which are to be transplanted after germination or after emergence from soil.

As used herein, the term “controlling” or “control” and similar terms refer to the ability of the compound of the subject matter to have a detrimental effect on the mollusc such as by preventing, combating, eradicating, destroying, repelling molluscs, increasing the mortality, inhibiting or reducing the growth and/or development of molluscs, paralysis, feeding inhibition and killing of molluscs. It may also include protecting plants from mollusc infestation and/or damage.

As used herein, the term “mollusc” refers to gastropod molluscs like terrestrial animals such as slugs, snails, etc., aquatic or marine animals such as mud snails, barnacles, etc.

As used herein, the term “effective amount” refers to an amount of the compound that, when ingested, contacted with or sensed, is sufficient to achieve a good level of control.

As used herein, the term “mixture” or “combination” refers, but is not limited to, a combination in any physical form, e.g., blend, solution, alloy, or the like.

As used herein, the term “post-emergence,” refers to the application of the molluscicide composition to the plants that have emerged from the soil. The term “pre-emergence” refers to the application of the molluscicide composition to a habitat, or soil, prior to the emergence of the plants from the soil.

The term “a” or “an” as used herein includes the singular and the plural, unless specifically stated otherwise. Therefore, the terms “a,” “an” or “at least one” can be used interchangeably in this application.

Throughout the application, descriptions of various embodiments use the term “comprising”; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

It was found that a mollusc population may be repelled and/or controlled by applying to the plant or to the soil in which the plants are planted an effective amount of a compound of Formula (I)

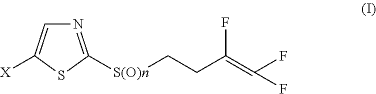

wherein
X represents halogen, and n represents 0, 1 or 2.

In some embodiments, X represents fluoro, chloro or bromo, and n represents 0 or 2. In a further embodiment, X represents chloro or bromo, and n represents 2. In a specific embodiment, the compound is fluensulfone when X represents chloro, and n represents 2.

In one embodiment, the present subject matter relates to the use of a compound of Formula (I) as defined hereinabove for controlling and/or repelling a mollusc population on plants or soil in which the plants are planted.

In specific embodiments, the plants or soil in which the plants are planted are infested with said molluscs.

In certain embodiments, the compound of Formula (I) as defined hereinabove may also be used to protect stored products from molluscs.

The effective application rates of the compound of Formula (I) cannot generally be defined, as it varies depending upon various conditions such as the type of the formulation, weather conditions and the type of crop.

The application rates of the compound of Formula (I) may vary depending on the desired effect. In an embodiment, depending on the desired effect, the application rates according to the present subject matter may be from approximately 1 g/ha to approximately 5000 g/ha. In another embodiment the application rates according to the present subject matter may be from approximately 1000 g/ha to approximately 4000 g/ha. In a specific embodiment, the application rates according to the present subject matter may be 2000 g/ha. The optimal rate for a specific application will depend on the crop and may be readily determined by established biological tests known to those skilled in the art.

The present subject matter further relates to a mollusc repelling composition comprising a repellent amount of a compound of Formula (I) as defined hereinabove. In a specific embodiment the composition may further include an agriculturally acceptable carrier.

In another embodiment, the composition may include at least one additional component selected from the group of surfactants, solid diluents, liquid diluents and other additives.

The active compounds according to the invention, as such or in their formulations, can also be used in a mixture with known herbicides, fungicides, bactericides, acaricides, nematicides or insecticides, plant growth regulators, biostimulants to widen, for example, the activity spectrum or to prevent the development of resistance. In many cases, this results in synergistic effects, i.e. the activity of the mixture exceeds the activity of the individual components. However, for the avoidance of doubt it is understood that such additional crop protection agents are unnecessary to achieve the effects of the present combinations. Accordingly, the present mollusc repelling composition may be limited to containing a compound of Formula (I) as the only crop protection agents.

The present compositions can be made at the time of use, or diluted. The present compositions can also be concentrated compositions, or so-called “ready-to-use” compositions, that is to say, compositions ready for use.

The present composition may be employed or prepared in any conventional form, for example, in the form of a twin pack, or for example, as wettable powders (WP), emulsion concentrates (EC), microemulsion concentrates (MEC), water-soluble powders (SP), water-soluble concentrates (SL), suspoemulsion (SE), oil dispersions (OD), concentrated emulsions (BW) such as oil-in-water and water-in-oil emulsions, sprayable solutions or emulsions, capsule suspensions (CS), suspension concentrates (SC), dusts (DP), oil-miscible solutions (OL), seed-dressing products, granules (GR) in the form of microgranules, spray granules, coated granules and absorption granules, granules for soil application or broadcasting, water-soluble granules (SG), water-dispersible granules (WDG), ULV formulations, microcapsules or waxes. These individual formulation types are known in the art.

Such compositions can be formulated using agriculturally acceptable carriers, surfactants or other application-promoting adjuvants customarily employed in formulation technology and formulation techniques that are known in the art.

Examples of suitable liquid carriers potentially useful in the present compositions include but are not limited to water; aromatic hydrocarbons such as alkylbenzenes and alkylnaphthalenes; chlorinated aromatics and chlorinated aliphatic hydrocarbons, such as chlorobenzene, chloroethylenes or methylene chloride; alcohols such as methanol, cyclohexanol, and decanol; ethylene glycol; polypropylene glycol; dipropropylene glycol; N,N-dimethylformamide; dimethylsulfoxide; N-alkylpyrrolidones such as N-methyl-2-pyrrolidone; paraffins; various oils such as olive, castor, linseed, tung, sesame, corn, peanut, cotton-seed, soybean, rape-seed, or coconut oil; fatty acid esters; ketones such as acetone, methyl ethyl ketone, methyl isobutyl ketone, cyclohexanone, 2-heptanone, isophorone, and 4-hydroxy-4-methyl-2-pentanone; and the like.

Examples of suitable solid carriers potentially useful in the present compositions include but are not limited to mineral earths such as silica gels, silicates, talc, kaolin, sericite, attaclay, limestone, bentonite, lime, chalk, bole, mirabilite, loess, clay, dolomite, zeolite, diatomaceous earth, calcium carbonate, calcium sulfate, magnesium sulfate, magnesium oxide, sodium carbonate and bicarbonate, and sodium sulfate; ground synthetic materials; fertilizers such as ammonium sulfate, ammonium phosphate, ammonium nitrate, ureas, and products of vegetable origin, such as cereal meal, tree bark meal, wood meal, and nutshell meal; cellulose powders; paper pulp; and other solid carriers.

Other ingredients, known as additives, such as wetting agents, anti-foaming, adhesives, neutralizers, thickeners, binders, sequestrates, fertilizers, biocides, stabilizers, buffers or anti-freeze agents, may also be added to the present compositions.

Surfactants may refer to any agriculturally acceptable material which imparts emulsifiability, stability, spreading, wetting, dispersibility, or other surface-modifying properties. Examples of suitable surfactants include, but are not limited to, non-ionic, anionic, cationic and ampholytic types such as alkoxylated fatty alcohols, ethoxylated polysorbate (e.g. tween 20), ethoxylated castor oil, lignin sulfonates, fatty acid sulfonates (e.g. lauryl sulfonate), phosphate esters such as phosphate esters of alcohol alkoxylates, phosphate esters of alkylphenol alkoxylates and phosphate esters of styrylphenol ethoxylates, condensates of sulfonated naphthalene and naphthalene derivatives with formaldehyde, condensates of naphthalene or of naphthalenesulfonic acid with phenol and formaldehyde, alkylarylsulfonates, ethoxylated alkylphenols and aryl phenols, polyalkylene glycols, sorbitol esters, alkali metal, sodium salts of lignosulphonates, tristyrylphenol ethoxylate phosphate esters, aliphatic alcohol ethoxylates, alkylphenol ethoxylates, ethylene oxide/propylene oxide block copolymers, graft copolymers and polyvinyl alcohol-vinyl acetate copolymers. Other surfactants known in the art may be used as desired.

Aqueous use forms can be prepared from emulsion concentrates, suspensions, pastes, wettable powders or water-dispersible granules by adding water. To prepare emulsions, pastes or oil dispersions, the components of the compositions either as such or dissolved in an oil or solvent, can be homogenized in water by means of a wetting agent, tackifier, dispersant or emulsifier. Alternatively, it is also possible to prepare concentrates comprising active ingredient, wetting agent, tackifier, dispersant or emulsifier and, if desired, solvent or oil, which are suitable for dilution with water.

The mollusc repelling composition can be applied pre-plant incorporated, pre- or post-emergence of plants.

The compound of Formula (I) as defined hereinabove may be employed in a customary manner by applying the active compound to the plants or to the environment of the plants that requires protection, for example, to the soil. The compound may be applied to the soil that is in proximity to the plants to be protected.

The compound of Formula (I) may be used to protect any agricultural crop, ornamental or vegetable plants from damage by molluscs. Suitable target crops for control of molluscs include but are not limited to, for example root and tuber vegetables (such as carrots, potatoes, radishes, sugar beets, or sweet potatoes); vegetables (such as onions, celery, lettuce, spinach, broccoli, cauliflower, cabbage, tomatoes, or peppers); legume vegetables (such as beans, peas or soybean); cucurbit vegetables (such cucumbers, melon, or squash); citrus fruits (such as oranges, tangerines, lemons, or grapefruits); fruit (such as apples, pears, cherries, peaches, plums, blackberries, raspberries, blueberries, elderberries, grapes, kiwi fruit, strawberries, almonds, or pecans); cereals (such as corn, rice, sorghum, wheat, barley, buckwheat, millet, oats, or rye); herbs and spices (such as basil, black pepper, or chive); oilseed crops (such as rapeseed, sunflower seed, soybean, safflower, canola, flax, or cottonseed) and sugar cane, hops, cotton, flax, alfalfa. The compound of Formula (I) may also be used to protect pasture grass, lawn grass, turf grasses, commercial turf, sports turf, sod, fragrant crops, ornamental plants, flowering plants, indoor plants, garden plants, garden trees and forest growths.

The compound of Formula (I) may be used to protect any areas in which mollusc infestation may occur. This may include but is not limited to agricultural fields, greenhouses, nurseries, public parks, public and private gardens, residential areas, golf courses, buffer strips in agricultural areas and forests.

The compound of Formula (I) of the present subject matter may be used to control and/or repel molluscs. For example, the present subject matter may be used to control and/or repel molluscs such as Agriolimax reticulatis, Ariolimax columbianus, Arion spp. (e.g. Arion ater, Arion circumscriptus, Arion distinctus, Arion fasciatus, Arion Flagellus, Arion hortensis, Arion intermedius, Arion rufus, Arion subfuscus, Arion silvaticus, Arion lusitanicus, Arion vulgaris), Bradybaena spp. (e.g. Bradybaena fruticum), Cantareus spp. (e.g. Cantareus aspersus), Cepaea spp. (e.g. Cepaea nemoralis, Cepaea hortensis), Cochlodina spp. (e.g. Cochlodina laminata), Deroceras spp. (e.g. Deroceras agrestes, Deroceras laeve, Deroceras panormitanum, Deroceras reticulatum), Discus spp. (e.g. Discus rotundatus), Euomphalia spp. (e.g. Euomphalia strigella), Galba spp. (e.g. Galba truncaluta), Helicigona spp., Arianta arbustorum, Helicella spp. (e.g. Helicella itala, Xenolenta obvia), Helicodiscus spp. (e.g. Helicodiscus panalellus), Helix spp. (e.g. Helix aperta, Helix aspensa, Helix pomatia, Helix nemoralis, Helix luconum), Limax spp. (e.g. Limax cineneonigen, Limax flavus, Limax manginatus, Limax maximus, Limax pinnae, Limax tenellus), Milax spp. (e.g. Milax gagates), Lymnaea spp., Opeas spp., Oxyloma spp. (e.g. Oxyloma pfeiffeni), and Pomacea spp. (e.g. Pomacea canaliculata), Tandonia spp. (e.g. Tandonia budapestensis, Tandonia sowerbyi), Vallonia spp., and Zonitoides spp. (e.g. Zonitoides nitidus, Zonitoides arboreus, Theba pisana, Subulina octona).

According to some embodiments, the mollusc controlling/repelling composition of the present subject matter may be applied in conjunction with other mollusc controlling/repelling agents and/or mollusc controlling/repelling mechanisms. Examples include fentin acetate, metaldehyde, methiocarb, niclosamid, thiodicarb and trimethacarb.

In another embodiment, the present subject matter provides a kit comprising the mollusk repelling composition as described herein, or components thereof. Such kits may comprise, in addition to the aforementioned active components, one or more additional active and/or inactive ingredients, either within the provided composition or separately.

### EXPERIMENTAL DETAILS

A split box trial was conducted using Deroceras reticulatum as the slug species and lettuce seedling as the food source.

There were 5 split treatments with each treatment replicated four times using a simple randomized block design. Each plot was a single enclosed cage of dimensions 0.6 m×0.4 m. Each cage was of opaque plastic construction with rigid sides and a removable fine muslin top to prevent escape of the slugs. The cages were each filled with natural loam soil to a depth of approximately 7 cm, which was consolidated to prevent slug escape.

Half of each box was covered with a polythene sheet before spraying to prevent the spray from reaching the soil on half of the box. The half of the box which received direct application of fluensulfone 250 CS may be defined as side X and the half of the box which was covered and did not receive direct spraying of fluensulfone 250 CS may be defined as side Y. Although fluensulfone was not directly applied to side Y, trace amounts of fluensulfone diffused to side Y. As such, effects of fluensulfone were observed in side Y as well. Following the application of fluensulfone 250 CS, one treatment was incorporated into the soil by mixing the top 2 cm of the soil. The other applications had no soil incorporation. Lettuce plants were planted into the soil either seven or two days after spraying. At these intervals after spraying, six lettuce plants were planted into each cage, three in the treated side and three in the untreated side. After transplanting the lettuce plants, eight adult slugs were introduced into the centre of each cage. The soil was kept damp for the duration of the trials. Treatment details are given in Table 1.

TABLE 1
Test Items and Treatments Applied

| | | Application | | |
|---|---|---|---|---|
| | | Rate/ha | | |
| Treatment | Product | liter | ai (gram) | Cultivation |
| 1 | Untreated | — | — | |
| 2 | Side X | 8.0 | 2000 | Soil incorporation-7 days |
| | Side Y | — | — | before planting lettuces |
| 3 | Side X | 8.0 | 2000 | No incorporation-soil |
| | Side Y | — | — | sprayed 7 days before |
| | | | | planting lettuces |
| 4 | Side X | 8.0 | 2000 | No incorporation-soil |
| | Side Y | — | — | sprayed 2 days before |
| | | | | planting lettuces |

All sprayed treatments applied in 200 l/ha water.

Assessments were carried out after the introduction of the slugs. Severity of leaf damage was assessed using a % scale where 0=no damage and 100=total loss of plant foliage. Incidence of plant damage was assessed by counting the actual number of plants showing damage.

TABLE 2
% of lettuce plants per plot which were damaged

| | | | 1 day after | 2 days after | 6 days after | 9 days after | 16 days after |
|---|---|---|---|---|---|---|---|
| Treatment | | Application | slug | slug | slug | slug | slug |
| No. | | Rate | introduction | introduction | introduction | introduction | introduction |
| 1 | Untreated | — | 21.7 | 41.7 | 38.3 | 58.3 | 75 |
| 2 | Side X | 8 L/ha | 5.0 | 13.3 | 8.3 | 13.3 | 13.3 |
| | Side Y | — | 5.0 | 13.3 | 13.3 | 33.3 | 16.7 |
| 3 | Side X | 8 L/ha | 8.3 | 5.0 | 0.0 | 8.3 | 0.0 |
| | Side Y | — | 13.3 | 21.7 | 21.7 | 33.3 | 21.7 |
| 4 | Side X | 8 L/ha | 0.0 | 13.3 | 13.3 | 16.7 | 5.0 |
| | Side Y | — | 5.0 | 13.3 | 21.7 | 30.0 | 8.3 |

When live slugs were assessed, fluensulfone appeared to have a repellent effect. In these treated boxes, the slugs tended to avoid the sprayed side of the box, and migrate to the untreated side. Initially, slugs were found in both sides of the boxes, but as the trial progressed, they migrated to the untreated side. Fewer live slugs were found overall in both sides of these boxes, due to the fact that the slugs burrowed under the soil surface compared to untreated boxes.

In all fluensulfone treated boxes, the numbers of damaged lettuce plants were reduced compared to the untreated box. It can be shown from the above tables that fluensulfone not only affected the treated side of the treated boxes, reduced levels of plant damage were also seen on the untreated side of the box. Due to diffusion of fluensulfone, slugs were repelled from both sides of the treated boxes. The repellent effect was such that slugs burrowed under the soil in both sides of the boxes. The amount of damage caused to the lettuce plants (damage severity) were reduced in the treated boxes when compared to the untreated box. Soil incorporation or time before transplanting did not have a significant effect on slug repellency.

While the present subject matter has been shown and described with reference to preferred embodiments thereof, it will be understood by those skilled in the art that many alternatives, modifications and variations may be made thereto without departing from the spirit and scope thereof. Accordingly, it is intended to embrace all such alternatives, modifications, and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

1-25. (canceled)

26. A method of controlling or repelling molluscs comprising applying to a plant or soil in which the plants are planted an effective amount of a compound of Formula (I) 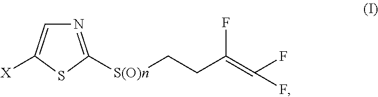
wherein
X represents halogen, and n represents 0, 1 or 2.

27. The method according to claim 26, wherein X represents fluoro, chloro or bromo, and n represents 0 or 2.

28. The method according to claim 26, wherein X represents chloro or bromo, and n represents 2.

29. The method according to claim 26, wherein X represents chloro.

30. The method according to claim 29, wherein n represents 0 or 2.

31. The method according to claim 29, wherein n represents 2.

32. The method according to claim 26, wherein the application rate is from 1 g/ha to 5000 g/ha.

33. The method according to claim 32, wherein the application rate is from 1000 g/ha to 4000 g/ha.

34. The method according to claim 26, wherein the plant or the soil in which the plants are planted is infested with said molluscs.

35. The method according to claim 26, wherein the plant is an agricultural plant, a flower, grass, turf or an ornamental plant.

36. The method according to claim 32, wherein the plant is an agricultural plant, a flower, grass, turf or an ornamental plant.

37. The method according to claim 34, wherein the plant is an agricultural plant, a flower, grass, turf or an ornamental plant.

38. The method according to claim 26, wherein the mollusc is a terrestrial mollusc.

39. A mollusc repelling composition comprising an effective amount of a compound of Formula (I) 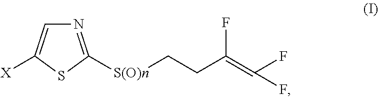
wherein X represents halogen, and n represents 0, 1 or 2.

40. The composition according to claim 39, wherein X represents fluoro, chloro or bromo, and n represents 0 or 2.

41. The composition according to claim 39, wherein X represents chloro or bromo, and n represents 2.

42. The composition according to claim 39, wherein X represents chloro.

43. The composition according to claim 42, wherein n represents 0 or 2.

44. The composition according to claim 43, wherein n represents 2.

45. The composition according to claim 39, further comprising an agriculturally acceptable carrier.

46. The composition according to claim 39, further comprising at least one surfactant, solid diluent, liquid diluent, other additives, or a combination thereof.

47. The composition according to claim 39, wherein the mollusc is a terrestrial mollusc.

48. A composition comprising an effective amount of a compound of Formula (I) 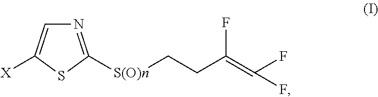
for use in controlling a mollusc population;
wherein X represents halogen, and n represents 0, 1 or 2.

49. The composition according to claim 48, wherein X represents fluoro, chloro or bromo, and n represents 0 or 2.

50. The composition according to claim 48, wherein X represents chloro or bromo, and n represents 2.

51. The composition according to claim 48, wherein X represents chloro.

52. The composition according to claim 51, wherein n represents 0 or 2.

53. The composition according to claim 52, wherein n represents 2.

54. The composition according to claim 48, further comprising an agriculturally acceptable carrier.

55. The composition according to claim 48, further comprising at least one surfactant, solid diluent, liquid diluent, or a combination thereof.

56. The composition according to claim 48, wherein the mollusc population is present in plants or soil in which the plants are planted.

57. The composition according to claim 56, wherein the plant is an agricultural plant, a flower, grass, turf or an ornamental plant.

58. The composition according to claim 48, wherein the mollusc is a terrestrial mollusc.

59. A kit comprising the composition according to claim 39.

60. A kit comprising the composition according to claim 48.
